(19) United States
(12) Patent Application Publication
(10) Pub. No.: US 2005/0090011 A1
(43) Pub. Date: Apr. 28, 2005

(54) Spectral analysis apparatus for measuring intermediate layers of mineral samplers

(75) Inventor: Francois Eberhardt Du Plessis, Stellenbosch (ZA)

Correspondence Address: CONNOLLY BOVE LODGE & HUTZ LLP, SUITE 800, 1990 M STREET NW, WASHINGTON, DC, 20036-3425, US

(73) Assignee: Blue Cube Intellectual Property Company (PTY) LT, Strand (ZA)

(21) Appl. No.: 10/502,300

(22) PCT Filed: Jan. 22, 2003
(86) PCT No.: PCT/ZA03/00011

(30) Foreign Application Priority Data
Jan. 23, 2002 (ZA) 2002/0578
Jan. 23, 2002 (ZA) 2002/0579
Jun. 21, 2002 (ZA) 2002/5000

(51) Int. Cl. G01N 33/20

(52) U.S. Cl. 436/73; 436/164; 422/82.05

(57) ABSTRACT

A method of spectral analysis of a mineral (M) in granular form is disclosed. The mineral (M) is moved through an illumination zone (18) and a beam of light is directed at the mineral (M) to illuminate it. Light reflected off the mineral (M) is collected and then spectrally analysed to obtain information pertaining to the composition of the granular material (M). The mineral (M) is in the form of a layer having an undersurface (20) and a top surface (22). The illumination zone (18) is intermediate the undersurface (20) and the top surface (22).

### FIELD OF THE INVENTION

This invention relates to mineral analysis.

### BACKGROUND TO THE INVENTION

In the metallurgical industry there is a need to know as accurately as possible the composition of the material that has been mined as sand, or mined as rock and then crushed to granular form, and constitutes the feedstock for a processing plant.

The main reason why this information on composition is of vital importance is that the way in which the various operating parameters of the minerals processing plant have to be set depends on the proportions of the constituents in the feedstock. These proportions vary continually as mining progresses through the body of ore being mined, and adjustments thus have to be made to ensure that the plant is being run at maximum efficiency to achieve the best possible efficiency.

Another reason why this information is of importance is that changes occur in environmental variables such as humidity and temperature, as well as in equipment variables such as supply voltage and physical condition. Any of these variables may cause certain stages of the process not to produce optimum intermediate streams of minerals. Ideally these error conditions have to be detected and corrected as soon as possible. Therefore up-to-date information on the composition of the mineral being processed is required to ensure that the plant is being run at maximum efficiency.

This information needed for these purposes is obtained by analysing the mineral being processed at various stages of its treatment. The proportions of the constituents as the mineral passes through the processing plant can be used as an indication of the efficiency of the plant and enable adjustments to the process to be made to achieve maximum benefication yield.

Because of a lack of accurate, up-to-date knowledge on the composition of the mineral, and the fear that valuables may be dumped with the discard, plant operators adopt a conservative approach and recycle a proportion of the mineral emerging from some or all of the stages to ensure that valuables are not lost. If accurate real time knowledge on the composition of the mineral being treated is available, recycling can be reduced without loss of valuable products.

The known methods of determining the proportions of the constituents in the minerals undergoing processing can be divided into “human” and “machine”. The main human method involves the preparation of a sample the proportions of the constituents of which are the same as the proportions in the mineral being processed. Obtaining the sample is a lengthy procedure. The first step is to take a number of kilograms of the granular mineral to ensure that the constituents of the sample are as representative as possible of the constituents of the bulk. The sample is split into small portions and then some of these portions recombined until the procedure has eliminated any remaining difference between the proportions in the sample and the proportions in the mineral being processed. The grains in the sample are then identified and counted by a skilled grain counter using a microscope and a grid over which the grains are spread.

Another “human” method relies on the skill of the person carrying out the test as it is based on the difference between the colour of the sample being checked and the colour of a standard sample. This is not a particularly accurate method as the human eye cannot pick up small changes in contrast or colour.

The “machine” methods are numerous but these have a number of shortcomings. Some require expensive equipment and generally sample preparation is time consuming. Also, some of the equipment is such that it can only be run by trained scientists and then only in laboratory conditions. Examples of known machine methods are:

Gravity Method

The sample is dropped into a heavy liquid, which is itself expensive, and which has a specific gravity of about 3. Most minerals have specific gravities of about 4 whereas silica has a specific gravity of approximately 2.5. Consequently separation occurs when the minerals sink and the silica floats. The separated grains are then further separated, counted and weighed.

Magnetic Method

Granular material which exhibits magnetic properties can be separated from granular material which is not magnetic by applying a magnetic field. This is followed by counting and weighing.

Electrostatic Method

Separation relies on the application of a static charge to separate granular material which is electrically insulating from that which conducts electricity.

Conductor/Non-Conductor Method

This is a recently developed technique which has not, to Applicant's knowledge and belief, been used commercially. It involves the application of a voltage to the sample and the detection of the leakage current which varies with composition.

X-Ray Fluorescence Method

When irradiated with X-rays different minerals fluoresce in different ways. This method, again requiring expensive equipment, gives a sub-set of which of the 92 naturally occurring elements are present and their proportions. It cannot determine how the actual mineral phases in the sample, or the elements, are bound to one another.

Induced Plasma Spectrometry

The sample is heated to a temperature such that it dissociates into a plasma. The radiation or absorption of light at different wavelengths is determined by the elemental composition of the plasma and this can be used to detect the constituents in the sample. This method only identifies the elements present and not the mineral phases.

X-Ray Diffraction

X-rays are directed at a piece of the ore. As they pass through they are diffracted, and the nature of the diffraction is determined by the composition of the ore. This is an empirical method but does provide information about the mineral phases.

Chemical Analysis Method

A sample is dissolved in a solvent and the methods of analytical chemistry applied to determine composition.

Spectral Method

The analysis of the reflection spectra from the polished surface of a crystal, from the side surface of a granular sample in a square sectioned stationary Cuvette, from the top surface of a compressed pill of the granular mineral and from the top surface of granular material in a rotating Petri dish have all been tried. The analysis of the absorption spectra of a liquid with the granular sample dispersed in it and the analysis of the absorption spectra of a wafer of the mineral have also been tried. All these methods require that a sample suitable for use in the method be prepared.

The preparation of samples for these methods is time consuming. Cutting and polishing of a crystal, or the preparation of a wafer, are pre-requisites to by the long method of splitting and recombining to ensure that the composition of the sample is representative of the composition of the bulk material.

Despite all the equipment and methods available Applicant is not aware of any spectral analysis apparatus that is used in the processing plant close to the stream of mineral being processed, and which can, in a time period measured in seconds rather than minutes, provide information on the constituents of the mineral in the stream.

The present invention seeks to provide a new analysis method and new analysis apparatus which enable information on the composition of the mineral under test to be obtained for process control purposes.

### BRIEF DESCRIPTION OF THE INVENTION

According to one aspect of the present invention there is provided a method of analysing a mineral in granular form to provide information pertaining to its composition, the method comprising moving said granular mineral through an illumination zone, directing a beam of light at said mineral to illuminate it, collecting light reflected from said granular mineral and spectrally analysing the reflected light to obtain information pertaining to the composition of the granular mineral, said mineral being in the form of a layer having an undersurface and a top surface, said illumination zone being intermediate the undersurface and the top surface of the layer.

In one embodiment of the method a probe is inserted into said layer so that a leading end of the probe is intermediate said undersurface and said top surface, the material in the vicinity of said leading end of said probe is illuminated, and light from the illuminated granular mineral in the vicinity of said leading end collected.

Preferably said probe is inserted into said granular mineral from above.

The method can include the step of scraping said layer so as to cause an upper part of the layer to be diverted so that it passes around said illumination zone thereby exposing, as an upwardly facing surface intermediate said undersurface and said top surface, granular mineral which was previously lying in said layer at a level between said upwardly facing surface and said top surface.

In a further embodiment of the method, the granular mineral is dispersed in a liquid to form a slurry which flows along a pipe having walling which constrains the slurry and causes it to flow axially along the pipe, inserting a probe into said pipe so that a leading end of the probe is spaced from said walling, illuminating the dispersed granules flowing in said pipe in an illumination zone spaced inwardly from said walling, and collecting light reflected from the granules in the slurry as they move through the illumination zone.

This embodiment of the method can comprise the step of positioning said leading end on the longitudinal axis of said pipe so that said illumination zone is on the axis of the pipe.

Another embodiment of the method comprises placing a layer of granular mineral in a vertically elongate container having a transparent wall, directing a beam of light at said wall to form an illumination zone intermediate the top surface of the powdered mineral and the undersurface of the granular mineral, rotating said container about a vertical axis so that the granular mineral in the container at a level intermediate said top and undersurfaces moves through said illumination zone, and collecting light reflected off the granular mineral in said zone.

This latter embodiment can include the step of directing said beam of light obliquely at said container wall so that light which reflects off the container wall is not collected for spectral analysis.

According to a further aspect of the present invention there is provided apparatus for analysing a mineral in granular form to provide information pertaining to its composition, the apparatus comprising a light source at an illumination zone for directing light onto said granular mineral at said zone, means for collecting light reflected from the granular mineral in said zone, means for spectrally analysing the reflected light, and means which constrains said granular mineral into a layer having an undersurface and a top surface, said illumination zone being at a level which is intermediate said top and undersurfaces of said layer.

One form of the apparatus includes a probe for insertion into said layer so that a leading end of the probe is at said illumination zone.

Preferably said light source is constituted by the end of a first optical fibre, the probe including a second optical fibre for transmitting reflected light to said means for spectrally analysing the reflected light.

To provide adequate light for illumination purposes and adequate light collecting ability the apparatus can include a group of first optical fibres and a group of second optical fibres.

The apparatus can include a scraper for causing an upper part of said layer to be diverted around the probe thereby to expose an upwardly facing granular mineral surface intermediate said undersurface and said top surface, said probe serving to illuminate, and to collect reflected light from, the intermediate granular mineral surface.

The invention provides a “bench top” model of the apparatus which comprises a vertically elongate container for receiving mineral in granular form, said container having transparent side walling and there being means for rotating said container about a vertical axis, said light source being positioned to direct light at the container to create an illumination zone intermediate the top and bottom surfaces of a charge of granular mineral in said container.

In this form said probe is preferably positioned so that light is directed obliquely at said container's walling thereby to prevent light reflected off said container being transmitted to said means for spectrally analysing the reflected light.

The present invention also provides an installation comprising apparatus including a probe as described above and a pipe along which a slurry comprising a liquid with mineral in granular form dispersed in it flows, said pipe including walling and said probe being inserted into said pipe through said walling thereof so that the leading end of the probe is within the pipe and spaced inwardly from the walling. In this form that surface of the leading end of the probe through which light passes faces upstream and slopes in the downstream direction from its lower edge towards its upper edge. Said surface preferably slopes at an angle of between 30° and 60° with respect to vertical.

The present invention further comprises an installation comprising apparatus including a scraper as described above and a conveyor on which a stream of mineral in granular form is moved, said scraper protruding into said stream of material to divert an upper layer of said stream and expose said intermediate granular material surface.

### BRIEF DESCRIPTION OF THE DRAWINGS

For a better understanding of the present invention, and to show how the same may be carried into effect, reference will now be made, by way of example, to the accompanying drawings in which:—

FIG. 1 is a pictorial view of a first form of apparatus for analysing a mineral in granular form;

FIG. 2 is a transverse section on the line II-II of FIG. 4;

FIG. 3 is a top plan view of the apparatus of FIGS. 1 and 2;

FIG. 4 is a diagrammatic longitudinal section on the line IV-IV of FIG. 3;

FIG. 5 is a pictorial view of a further form of apparatus for analysing a mineral in slurry form;

FIG. 6 is a section on the line VI-VI of FIG. 8;

FIG. 7 is a top plan view of the apparatus of FIGS. 5 and 6;

FIG. 8 is a diagrammatic section on the line VII-VII of FIG. 7;

FIG. 9 is a pictorial view of yet another form of apparatus for analysing a mineral in granular form;

FIG. 10 is a section in the line X-X of FIG. 12;

FIG. 11 is a top plan view of the apparatus of FIGS. 9 and 10;

FIG. 12 is a side elevation of the apparatus of FIGS. 9 to 11;

FIG. 13 is a vertical section through yet another form of apparatus for analysing a mineral in granular form; and

FIG. 14 is a block diagram.

### DETAILED DESCRIPTION OF THE DRAWINGS

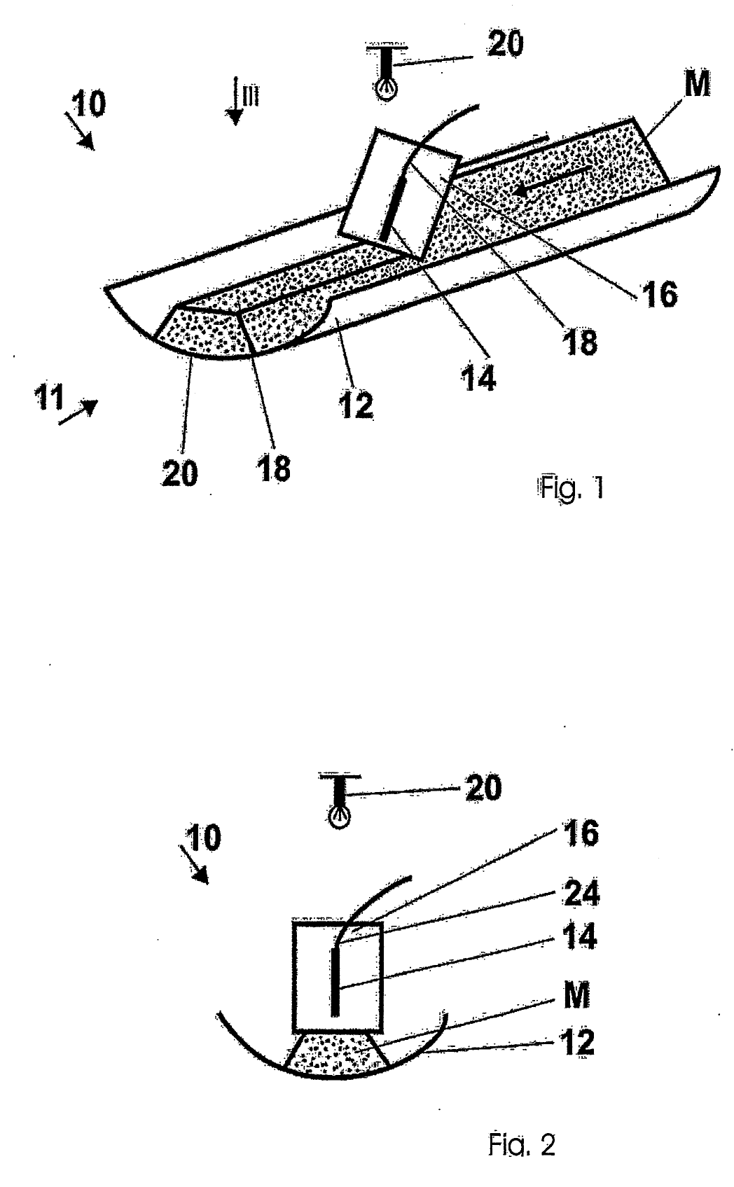

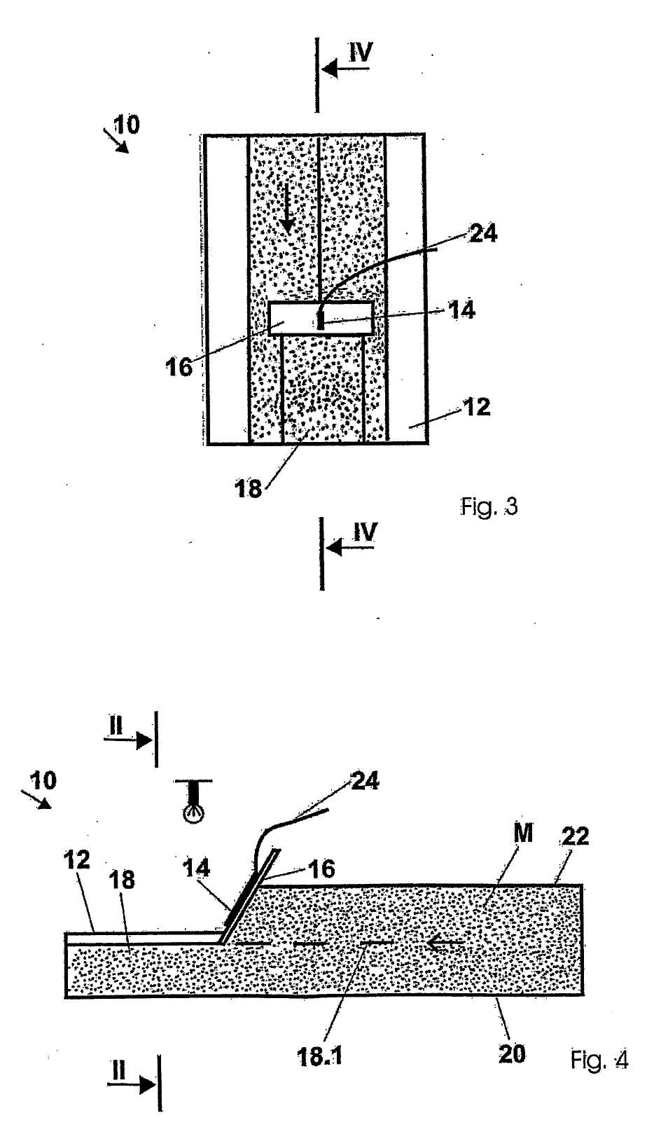

Referring firstly to FIGS. 1 to 4, the apparatus according to the invention for analysing a granular mineral forms part of an installation designed 10. The installation comprises a conveyor belt 12 which carries a stream of mineral M in granular form. The conveyor belt 12 can run from an ore crushing plant to the first stage of the processing plant, can be between stages of the processing plant or can be downstream of the last stage of the processing plant in which event it carries the valuables which have been recovered or it carries the residue which is being discarded. At all these stages a knowledge of the constituents of the granular mineral is of value to the plant operators as it enables them to control the parameters of the plant in a manner which leads to the greatest efficiency in operation.

The apparatus includes a probe 14 which is mounted on that face of a scraper 16 which faces downstream. As best seen in FIG. 4, the scraper 16 is inclined and has the effect of causing an upper part of the layer of granular mineral M to be diverted around the probe 14. This shields the probe 14 from the abrasive granular mineral and also has the effect of exposing an upwardly facing intermediate granular mineral surface which has been designated 18. The surface 18 is intermediate the undersurface 20 of the layer of material on the conveyor belt 12 and the top surface 22 of the layer. The surface 18 has been extended to the right in FIG. 4 as a dotted line 18.1 so as to indicate clearly the position of the surface 18 in the layer of material being conveyed.

The probe 14 comprises at least two, but preferably a multiplicity of, optical fibres 24 as will be described in more detail hereinafter with reference to FIG. 14. A first group of the optical fibres receives light from, for example, a tungsten halogen bulb or a pulsed xenon lamp. The light travels along the first group of optical fibres, and the tips of these fibres form a light source which illuminates the surface 18. The other group of fibres does not convey light to the probe 14 but receives light reflected from the surface 18 and transmits it to a spectrometer. This will also be described hereinafter with reference to FIG. 14.

By creating the intermediate surface 18, analysis takes place in a zone which most closely represents the overall composition of the crushed ore. In the uppermost and lowermost layers, vibration and other forces cause stratification and analysis of the material in these layers gives inaccurate results.

It is possible to illuminate the surface 18 directly by means of a tungsten halogen bulb or a pulsed xenon lamp 20 placed close to the surface 18. In this form only the group of fibres which collect reflected light and transmit it to the spectrometer are required.

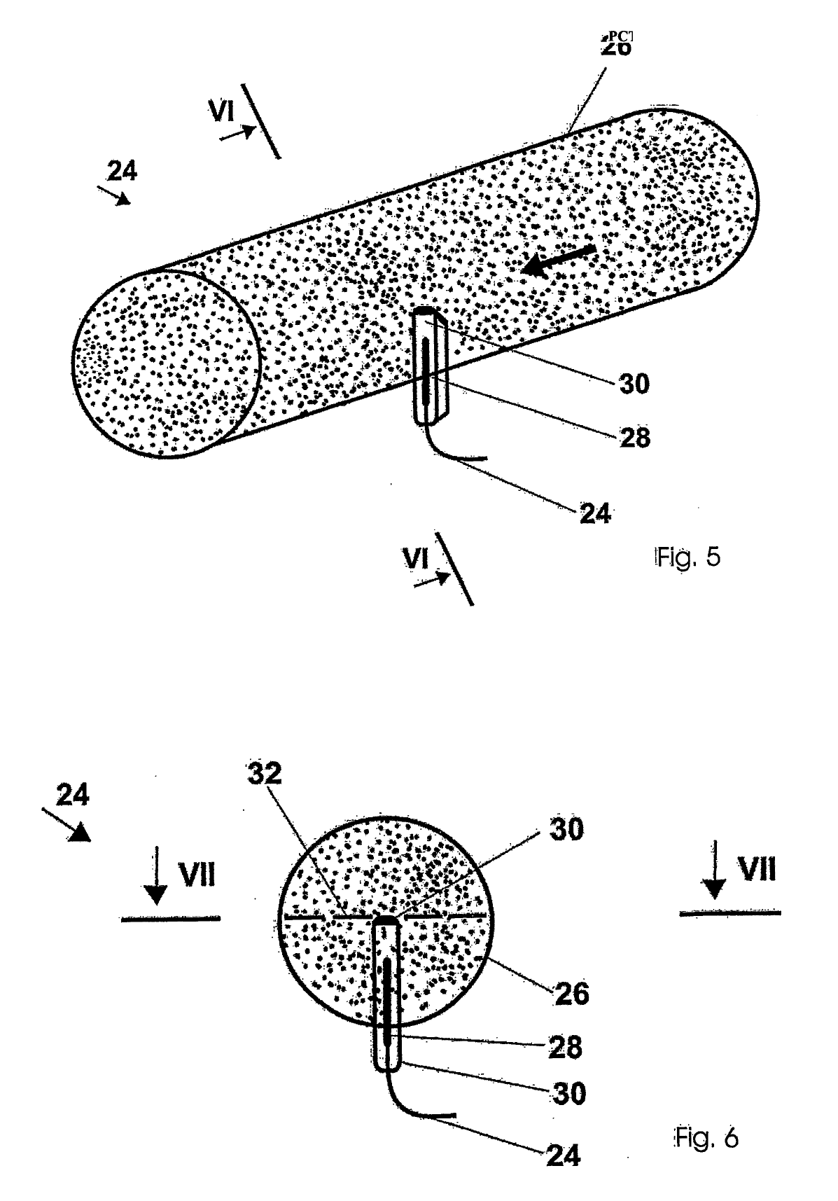

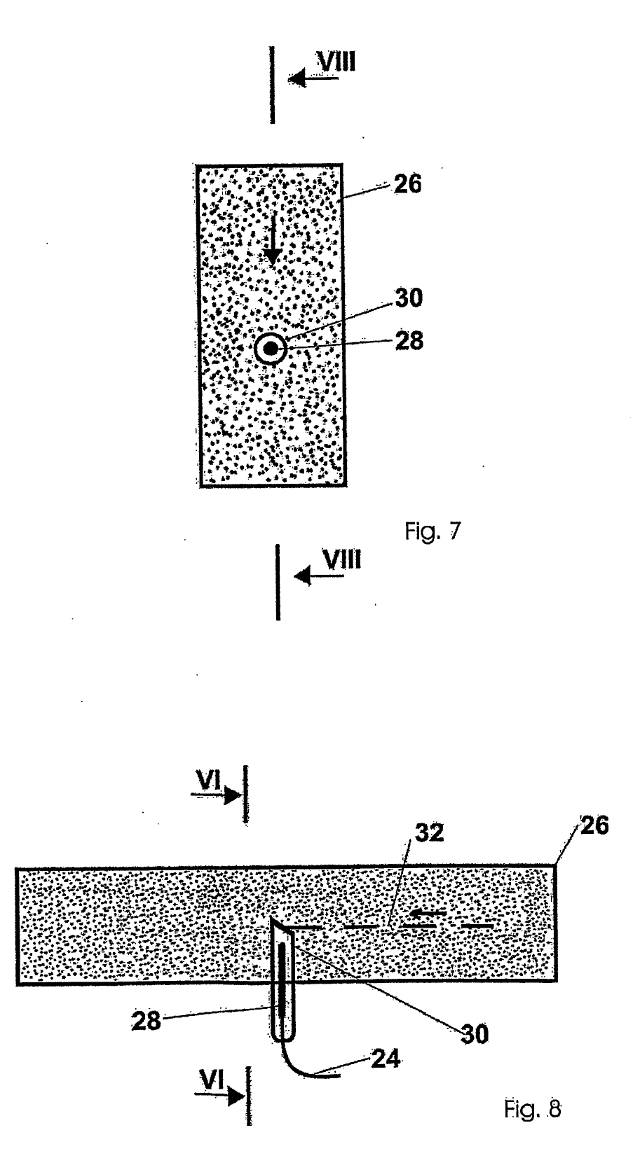

In the embodiment of FIGS. 5 to 8 the granular mineral is not in a dry form as in FIGS. 1 to 4 but is in the form of a slurry comprising a liquid with the mineral granules dispersed in it. The slurry flows along a pipe designated 26. In this form the probe is designated 28 and is within a protective housing 30. That surface of the upper end of the housing 30 (see particularly FIG. 8) through which light passes faces upstream and slopes in the downstream direction from its lower edge towards its upper edge. The angle of inclination can be between 30° and 600 with respect to horizontal. The reason for the angled surface is three fold. It prevents specular reflection of the source light directly from the glass surface back into the optical fibres, it enables a natural cleaning action of the glass window by the slurry, and it causes a non-laminar flow of the slurry over the surface. The inclined upper end of the housing 30 can be of a material such as sapphire which resists abrasion by the flowing slurry better than most other transparent materials. In this form the granular mineral flows as a layer the top surface of which lies beneath the upper part of the pipe and the undersurface which lies above the lower part of the pipe. The plane in which the surface which is illuminated by the probe 28 lies is designated 32 in FIGS. 6 and 8 and is intermediate the top and underlying surfaces of the flowing slurry.

The apparatus shown in FIGS. 5 to 8 operates in the same manner as that shown in FIGS. 1 to 4 and will be described hereinafter with reference to FIG. 14.

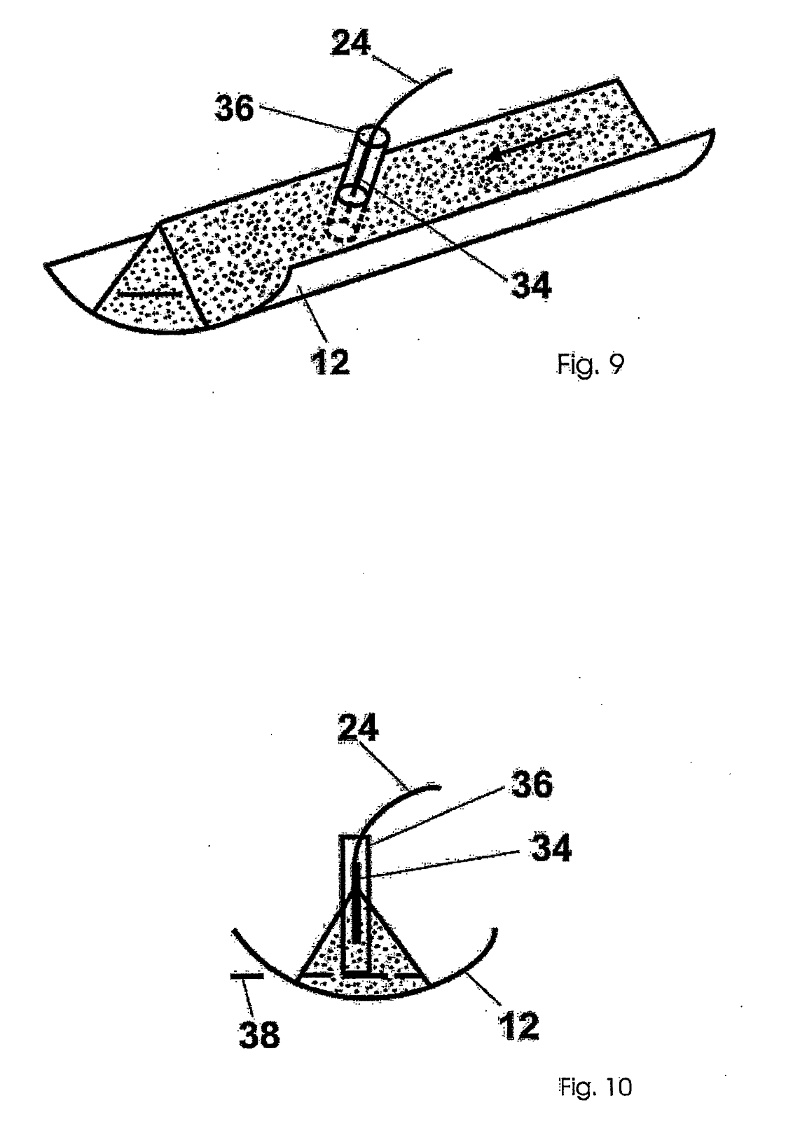

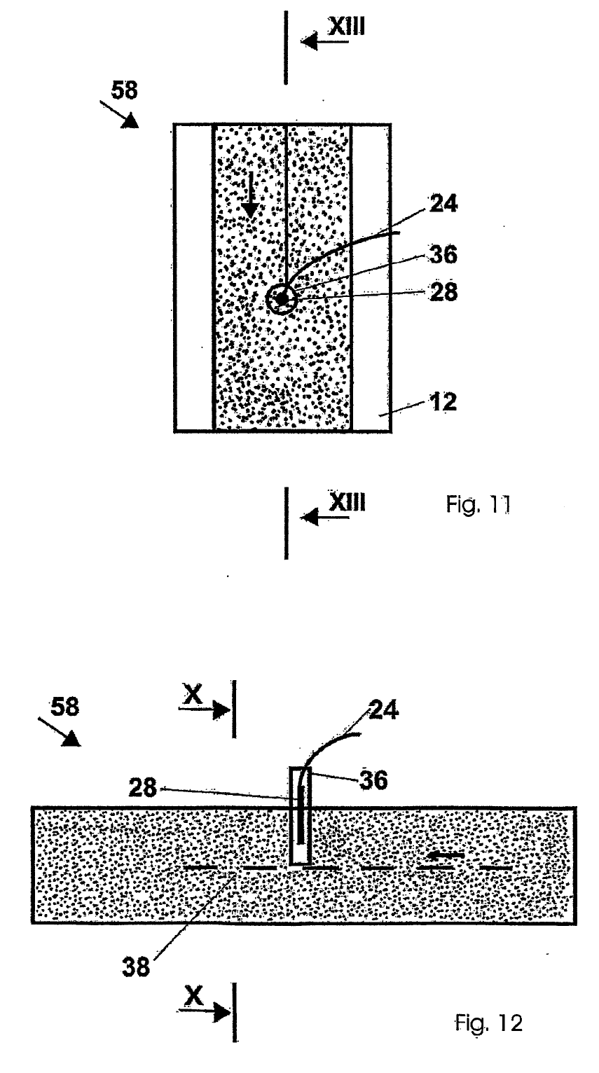

In the embodiment of FIGS. 9 to 12 the scraper 16 is omitted and the probe, designated 34, is within an abrasion resistant cylindrical casing 36. The tip of the probe can be set back from the end of the casing and is covered by a transparent portion of the casing. The position of the tip of the probe is such that it illuminates a zone of the granular mineral which is intermediate the undersurface of the layer, which is adjacent the conveyor belt 12, and the exposed top surface of the layer. The layer is, simply for the sake of illustration, shown as being approximately triangular in section. The illuminated surface lies in the plane designated 38 in FIGS. 10 and 12.

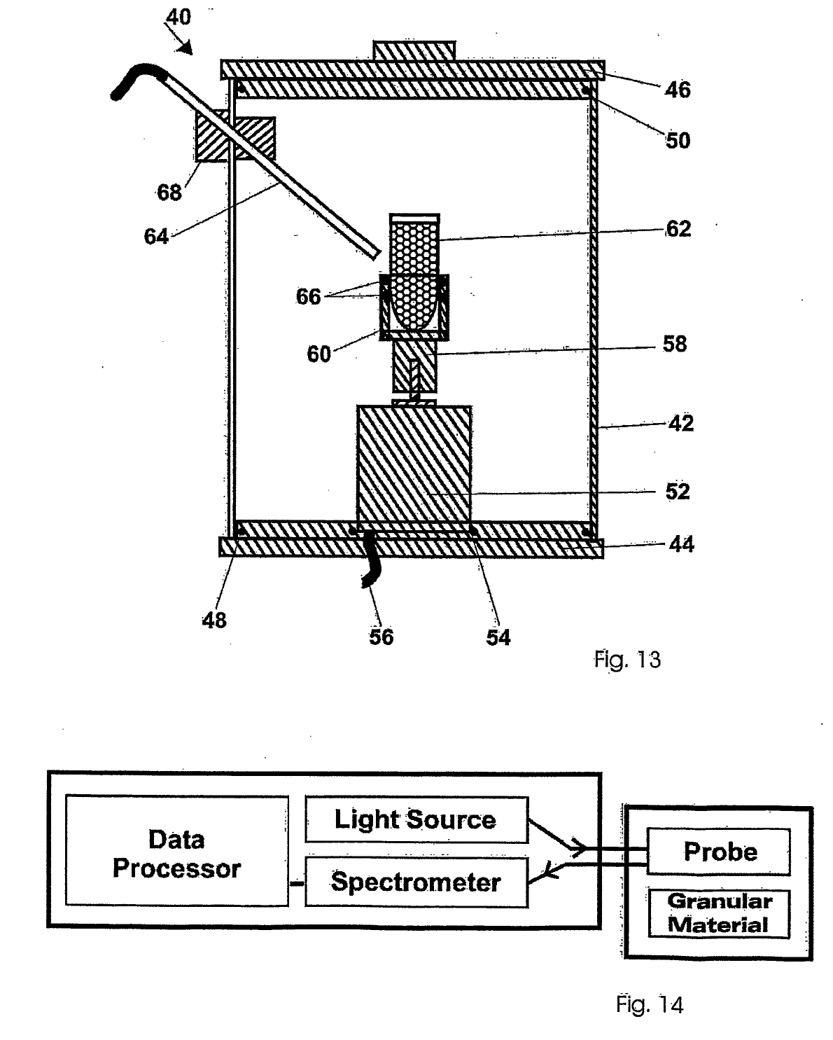

Turning now to FIG. 13, the illustrated apparatus for analysing a mineral in granular form is intended for “bench top” use in, for example, a laboratory or the control room of a minerals processing plant rather than as part of an installation as described above. The analysing apparatus shown in FIG. 13 is designated 40 and comprises a cylindrical housing 42 which can be of aluminium, a base 44 which can be of mild steel and a lid 46 which can be of PVC. The base and lid 44, 46 fit into the housing 42 in a manner which excludes ambient light from entering the housing 42, there being O-ring seals 48 and 50 between the base and the housing and the lid and the housing.

An electric motor and gearbox unit 52 is mounted in a socket 54 in the base 44. The power lead for the motor 50 is shown at 56.

A holder 58 is secured to the output shaft of the motor and gearbox unit 50, the holder 58 providing an upwardly open socket 60 for receiving a vertically elongate container 62 which receives the granular mineral to be analysed. The container 62 can be in the form of a glass or synthetic plastics material tube and can as a consequence be completely transparent. Alternatively, the container 62 can be of fabricated construction in which event a zone of its cylindrical side wall has to be transparent so that light from a probe 64 can shine on the granular mineral M in the container 62. O-rings 66 are provided in the socket 60 and grip the container 62 thereby preventing any movement thereof apart from rotation with the holder about its vertical, longitudinal axis.

A plug 68 fits in an opening provided therefor in the housing 42, the probe 64 entering the housing through the plug 68. As clearly illustrated, the probe 64 is at an angle, which can be between 30° and 60° with respect to the vertical, so that any light which reflects off the surface of the container 62 is not reflected back to those optical fibres which receive light reflected off the granular mineral. This prevents such reflected light from reaching the spectrometer.

Because the container 62 rotates, the illumination zone in this construction is an annular zone intermediate the top surface of the powdered mineral in the container 62 and the undersurface of the powdered mineral. The undersurface is constituted by the hemispherical bottom portion of the container 62.

Referring finally to FIG. 14, the halogen bulb or pulsed xenon lamp is designated 70 and the group of optical fibres leading t the probe is designated 72. The probe is designated 74 the group of optical fibres which collects reflected light and transmits it to the spectrometer 76 is designated 78. The spectrometer 76 is connected to a data processor 80 which is in turn connected to a monitor 82 on which the results of the analysis are displayed in graphical form.

To determine the proportions of two minerals A and B in a sample X containing a mixture these minerals in unknown proportions, it is necessary to obtain the spectra of each mineral in pure form and the spectra of the mixture X of unknown proportions.

In this example only five values from the spectra of each of A, B an X are used but in practice there may be many thousands of values each representing reflectance at a specific wavelength.

The example spectra are:

A: [0.2 0.2 0.2 0.6 1.4]
B: [1.4 0.6 1.0 1.4 1.0]
X: [0.5 0.3 0.3 0.9 1.3].
   Solution:

The example spectra show that there are differences between the reflectivity of A and B, which is a pre-requisite to use of this technique.

Even though two percentages are required, only one, for instance O_{A}, needs to be determined from the spectra, and because the unknown mixture contains only the two known minerals O_{B} can be calculated as:
O_{B}=100%−O_{A}
O_{A} can be determined from the spectra as follows:

$${O}_{A} = \frac{100 \%}{N} \times {\sum }_{j = 1}^{N} ( {X}_{j} - {A}_{j} ) / ( {B}_{j} - {A}_{j} )$$

, where Aⱼ represents the individual values from the spectrum of A, Bⱼ represents the individual values from the spectrum of B, Xⱼ represents the individual values from the spectrum of X, and N is equal to the number of samples per spectra, which in this case is 5.

Calculating for the example spectra provided in the problem statement yields the following answers:
O_{A}=25.0%
O_{B}=75.0%.

Therefore the sample X contains 25% of A and 75% of B.

If the mixture contains more than two minerals, then the spectra of each of the pure minerals is required together with the spectra of the mixture. Two or more unknowns must be calculated.

1. A method of analysing a mineral in a granular form to provide information pertaining to its composition, the method comprising moving said granular mineral through an illumination zone, directing a beam on light at said mineral to illuminate it, collecting light reflected from said granular mineral and spectrally analysing the reflected light to obtain information pertaining to the composition of the granular mineral, said mineral being in the form of a layer having an undersurface and a top surface, said illumination zone being intermediate the undersurface and the top surface of the layer.

2. A method as claimed in claim 1 and including the step of inserting a probe into said layer so that a leading end of the probe is intermediate said undersurface and said top surface, illuminating the material in the vicinity of said leading end of said probe, and collecting light from the illuminated granular mineral in the vicinity of said leading end.

3. (canceled)

4. A method as claimed in claims 1 or 2 and including the step of scraping said layer so as to cause an upper part of the layer to be diverted so that it passes around said illumination zone thereby exposing, as an upwardly facing surface intermediate said undersurface and said top surface, granular mineral which was previously lying in said layer at a level between said upwardly facing surface and said top surface.

5. A method as claimed in claim 1, wherein the granular mineral is dispersed in a liquid to form a slurry which flows along a pipe having walling which constrains the slurry and causes it to flow axially along the pipe, inserting a probe into said pipe so that a leading end of the probe is spaced from said walling, illuminating the dispersed granules flowing in said pipe in an illumination zone spaced inwardly from said walling, and collecting light reflected from the granules in the slurry as they move through the illumination zone.

6. (canceled)

7. A method as claimed in claim 1, and comprising placing a layer of granular mineral in a vertically elongate container having a transparent wall, directing a beam of light at said wall to form an illumination zone intermediate the top surface of the powdered mineral and the undersurface of the granular mineral, rotating said container about a vertical axis so that the granular mineral in the container at a level intermediate said top and undersurfaces moves through said illumination zone, and collecting light reflected off the granular mineral in said zone.

8. (canceled)

9. Apparatus for analysing a mineral in granular form to provide information pertaining to its composition, the apparatus comprising a light source at an illumination zone for directing light onto said granular mineral at said zone, means for collecting light reflected from the granular mineral in said zone, means for spectrally analysing the reflected light, and means which constrains said granular mineral into a layer having an undersurface and a top surface, said illumination zone being at a level which is intermediate said top and undersurfaces of said layer.

10. (canceled)

11. Apparatus as claimed in claim 9, wherein said light source is constituted by the end of a first optical fibre, and there is a second optical fibre for transmitting reflected light to said means for spectrally analysing the reflected light.

12. (canceled)

13. Apparatus as claimed in claim 11 and further including a scraper for causing an upper part of said layer to be diverted around the probe thereby to expose an upwardly facing granular mineral surface intermediate said undersurface and said top surface, said probe serving to illuminate, and to collect reflected light from, the intermediate granular mineral surface.

14. Apparatus as claimed in claim 11 or 13 and comprising a vertically elongate container for receiving mineral in granular form, said container having transparent side walling and there being means for rotating said container about a vertical axis, said light source being positioned to direct light at the container to create an illumination zone intermediate the top and bottom surfaces of a charge of granular mineral in said container.

15. (canceled)

16. An installation comprising apparatus as claimed in claim 11 or 13 and a pipe along which a slurry comprising a liquid with mineral in granular form dispersed in it flows, said pipe including walling and said probe being inserted into said pipe through said walling thereof so that the leading end of the probe is within the pipe and spaced inwardly from the walling.

17-19. (canceled)
